(19) United States
(12) Patent Application Publication
(10) Pub. No.: US 2005/0251080 A1
(43) Pub. Date: Nov. 10, 2005

(54) Magnetic vector control system

(76) Inventor: Edward Robert Hyde JR., Turlock, CA (US)

Correspondence Address: Edward R. Hyde, Jr., P.O. Box 3907, Turlock, CA, 95381, US

(21) Appl. No.: 11/123,811

(22) Filed: May 6, 2005

Related U.S. Application Data
(60) Provisional application No. 60/521,499, filed on May 6, 2004.

(51) Int. Cl. A61F 5/00

(52) U.S. Cl. 602/26

(57) ABSTRACT

Aging, injury and/or other pathologies of joints, especially weight bearing joints, contribute to changes in natural biomechanics. Deviations from optimal biomechanics lead to acceleration of the natural history of joint pathology and ultimately osteoarthritis. A Magnetic Vector Control System made up of an assembly of magnetic field sources can be disposed at or near a joint typically on or in adjacent bones of the joint, on one side of a first mechanical axis that creates a torque or moment about a second different axis of the joint, that intersects the first mechanical axis, to decrease the joint reactive force at the joint surface or equivalently substantially shift the first mechanical axis to a new or preferred position.

This application claims the benefit of provisional application MAGNETIC VECTOR CONTROL SYSTEM—No. 60/521,499 that was filed on May 6, 2004.

### BACKGROUND OF INVENTION

Treatment for joint pathologies usually beings well after symptoms reach substantial levels and the patient is experiencing pain and dysfunction.

Many times underlying pathologies are known prior to the onset of symptoms whether due to injury, congenital problems or acquired problems. These problems produce maladaptive biomechanics of a joint or an extremity segment and lead to dysfunction and pain. Osteoarthritis in joints occurs and is accelerated by improper biomechanics. Currently early treatments concentrate on physical therapy, bracing and assist devices. These treatments are directed towards decreasing symptoms and hopefully slowing the natural progression of the disease.

The improper biomechanics at the joint or segment can stem from structural, mechanical, motor, neurological or metabolic etiologies.

A joint can experience improper pathways in 6 Degrees Of Freedom (6DOF). Abnormal loads, abnormal moments, abnormal Instant Axis of Rotation (IAR) and abnormal centers of rotation (CR) can be present.

Methods that urge the joint or segment back towards proper alignment and function have been attempted. There are many non-surgical and surgical methods but their reliability and effectiveness is felt to be limited. These include braces, Ankle-Foot Orthoses (AFO), shoe wedges, etc. Offloading forces in joints that have already developed substantial osteoarthritis is accomplished by osteotomies. The High-Tibial Osteotomy is used in the knee to offload the medial compartment of the knee. Other procedures have been developed for other joints.

Controlled magnetic fields have been introduced into the field of orthopedics to treat bone and joint pathologies. (Hyde U.S. Pat. No. 6,387,096) Magnetic field interactions can be utilized to treat maladaptive biomechanics before pathologies develop or at least attenuate the speed of progression and/or ultimate level of pathology. They can also be used to offload joints that have already been destroyed by osteoarthritis.

This is done by using magnetic energy force vectors to correct or re-establish more normal biomechanics. The magnetic systems to correct biomechanics by the introduction of magnetic force vectors are called Magnetic Vector Control Systems (MVCS) MVCS with their associated method, instrumentation and implants can be used to address biomechanical disruption of any joint or body segment. The knee will be used as an example.

Additional magnetic force vectors are established by magnets or magnetic arrays and added to the intrinsic force vectors of the joint or system. Electromagnets and Magnetic induction can also be used to provide magnetic energy. These sources can be used independently or in combination with magnets or magnetic arrays. The added magnetic force vectors are used to shift the maladaptive forces caused by disruption of the normal biomechanics towards a more normal position or functional state. The magnetic force vectors can also be used to offload worn out areas of a joint.

### SUMMARY OF INVENTION

The knee joint will be used to demonstrate the invention. It is a very complex joint and has 6DOF.

The motions can occur in three planes. The planes are the coronal, the sagittal and the axial planes. The knee can rotate or translate in each plane. Motions in more than one plane can occur simultaneously.

The coronal plane will be considered here to describe the technology. The knee is thought of as having a weight bearing axis and a mechanical axis. It also has an axis of rotation in the sagittal plane, felt to be generally through the transepicondylar axis of the distal femur. This axis allows Adduction/Abduction of the knee. The knee can also be described as rotating in the coronal plane at a point near the medial intercondylar eminence, shifting weight from one compartment to the other. (Medial to Lateral) The weight-bearing axis (WBA) in single leg stance is felt to pass through the center of the femoral head of the hip joint, continuing through the knee joint at or near the medial intercondylar eminence and then pass through the middle of the ankle joint. The mechanical axis of the femur for a normal knee is generally in seven degrees of valgus with respect to the WBA. The mechanical axis of the tibia in a normal knee is in line with the WBA and perpendicular to the knee joint line.

A knee that is in varus or valgus from this aligned position will develop a moment at the point of rotation in the coronal plane. A varus knee will have an ADduction moment and a valgus knee will have an ABduction moment.

The ADduction moment in the varus knee will disrupt the normal balance between the Body Weight (BW) force vector, the compensatory muscle/ligament force vectors and the joint reaction force (JRF). The disruption in the normal biomechanics necessitates that a new equilibrium between the force vectors be established. Equilibrium is established by movement of the contact point between the femur and the tibia and an increase in the forces supplied by muscles and ligaments. The WBA is shifted medial to the knee by the varus alignment of the knee. The JRF is increased in magnitude and shifted more medial in the medial compartment. This creates an ADduction moment that is instrumental in the Varus Thrust that occurs in single leg stance phase. The Varus Thrust is an abrupt shift of a substantially neutral knee alignment in swing phase (non-weight bearing) to a varus alignment with the JRF shifted abruptly to the medial joint line.

The ADduction moment has to be balanced by the knee system to be in equilibrium, which requires an increase in the compensatory muscle force required and an increase the JRF in the new position in the medial compartment. A patient who can provide the compensatory muscle and ligamentous forces can balance or reduce the ADduction moment. The JRF however is still increased and still positioned to place most all of the JRF on the medial joint.

The addition of the new vector or ADduction moment changes the biomechanics of the knee dramatically and will lead to rapid loss of joint cartilage in the medial compartment and subsequent osteoarthritis because of localization of forces to the medial compartment.

Magnetic field interactions can be introduced as MVCS near or around the knee joint in this case. The MVCS can be implanted in or around the bones of the knee joint or attached to implants that are then attached to the bones.

These MVCS introduce substantially compensatory force vectors, which are placed such that they work to counteract the maladaptive forces and moments. (i.e. ADduction Moment, IAR, CR, etc.) The knee joint application will be restricted to the coronal plane. The invention can be applied and function in any plane or combinations of planes. The ADduction or ABduction moments cause varus or valgus motions of the tibia with respect to the femur.

A varus knee has an ADduction moment with a concomitant shift of the JRF to the medial compartment of the joint.

A MVCS is combination of magnetic energy sources that can be provided or supplied by permanent magnets, electromagnets or by magnetic induction or any combination of these sources. MVCS are placed at or near a joint and typically on or in adjacent bones of a joint. The MVCS units interact across the joint space in repulsion, attraction or combinations of attraction and repulsion. A MCVS is placed at the medial joint line in this example to create an ABduction moment provided by a substantially repulsive force between the two MVCS units. (Stabilizing forces to control shear of the magnetic units in repulsion can also be incorporated). This ABduction moment will help to counteract the ADuction moment.

An average size knee will be used in this example from an average size man (5″9″, 170 lbs). The ADduction moment for a varus knee has been measured to be about 4% of (BW×height)=(0.04)(170)(5.75)=39 ft-lbs. (Normal Knee=3.0+/−0.6%). The ABduction moment arm of the repulsive MVCS will be about 1.5 in (0.125 ft). Using the example of a repulsive force of 50 lbs between magnetic units the ABduction moment will be (0.125) (50)=6.25 ft-lbs. Another type of MVCS placed at the lateral joint line that provides a substantially attractive force. (Stabilizing forces can also be incorporated) creates another ABduction moment. This can be used alone or with a medial repulsive system. Using the example of an attractive force of 50 lbs, this MVCS will also generate an ABduction moment 6.25 ft-lbs.

Together the repulsive and the attractive ABduction moments (Force Couple) will provide 12.5 ft-lbs. of an ABduction moment. This reduces the ADduction moment by 12.5 lbs or 32%. Two MCVS that could provide 144 lbs each of attraction (lateral) and repulsion (medial) could completely cancel the ADduction moment caused by the varus misalignment. MCVS can be used on the medial and/or lateral sides independently or as a force couple where the medial and lateral MVCS synergistically act to restore normal biomechanics or act to offload the worn out area of the joint.

Complete cancellation of the ADduction moment is not necessary to relieve symptoms or to slow progression to osteoarthritis. The normal mechanics do need to be restored, however, to stop progression.

Stabilizing forces for repulsive MVCS to control shears that occur when two simple magnets are placed in repulsion can be accomplished readily by the use of Magnetic Arrays instead of plain magnets on the repulsive side.

Attractive MVCS are easier to control and construct. They can be made of simple magnets, hard magnetic and soft magnetic material combinations, electromagnets and/or magnetic induction systems. The MVCS can be made of any other combination or source of magnetic fields.

The previous example using the knee as an illustration has only been described in one plane, the coronal plane. MVCS or combinations of repulsive and attractive MVCS can be used in any number of planes. They can be used in the sagittal and/or axial planes as well or alone or in combination with coronal systems.

These can be used influence any vector or moment, as well as, the center of rotation, IAR or any vector system to make the maladaptive biomechanics return towards normal and in some cases be completely corrected.

### BRIEF DESCRIPTION OF DRAWINGS

1. ADduction Moment—Knee

2. Normal vs Varus Knee Force Vectors

3. Normal (Static), Varus (Static) & Normal (Dynamic) Force Vectors

4. ADduction, Flexion & Extension Moments

5. ADduction Moment Stabilizers

6. Axial Movement of Tibial Contact Points (Axial Plane)

7. AP Knee with Mechanical Axis and Weight-Bearing Axis

8. AP Knee with Normal Force Vectors

9. AP Knee with Normal Force Vectors (Only)

10. Varus Knee and ADduction Moment

11. AP Varus Knee and ADduction Moment with Vectors

12. AP Varus Knee and ADduction Moment with Vectors (Only)

13. AP Varus Knee and ADduction Moment with Vectors & MVCS

14. AP Varus Knee and ADduction Moment with Vectors & MVCS (Only)

15. AP Corrected Varus Knee and ADduction Moment with Vectors & MVCS

16. AP Corrected Varus Knee and ADduction Moment with Vectors & MVCS (Only)

17. AP Over-Corrected Varus Knee and ADduction Moment with Vectors & MVCS (Only)

18. Axial views of rod/screw shaped MCVS placed in the tibia

19. Rod MCVS placed through medial portals

20. Rod MVCS placed through anterior portals

21. MCVS placed through anterior portals

### DETAILED DESCRIPTION

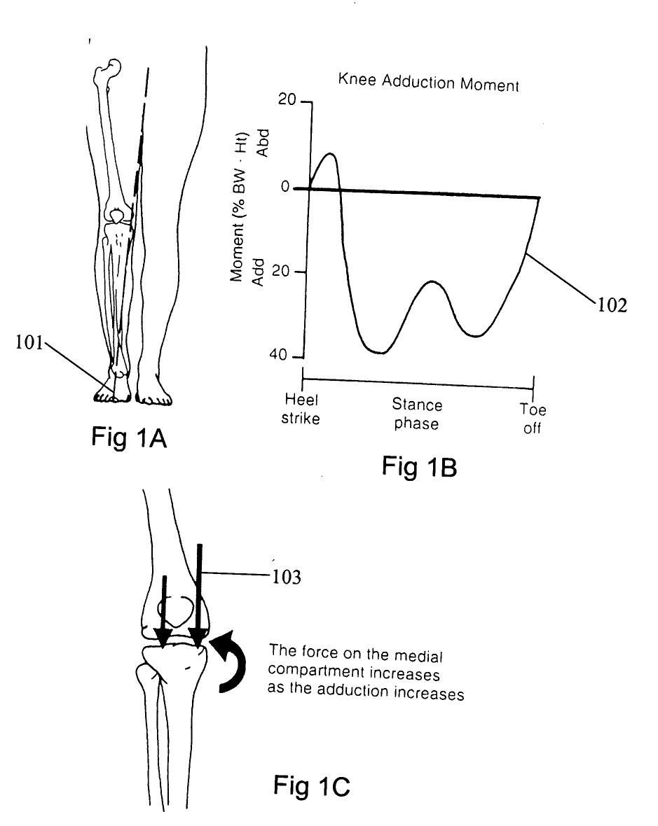

FIG. 1A shows a drawing depicting a weight bearing axis 101 that corresponds to a double leg stance. FIG. 1B is a representation of the single leg moments 102 in stance phase (weight bearing). These moments change throughout the stance phase from heel strike to toe off. Most of the moments are ADduction moments. These ADduction moments FIG. 1C 103 increase the force on the medial compartment.

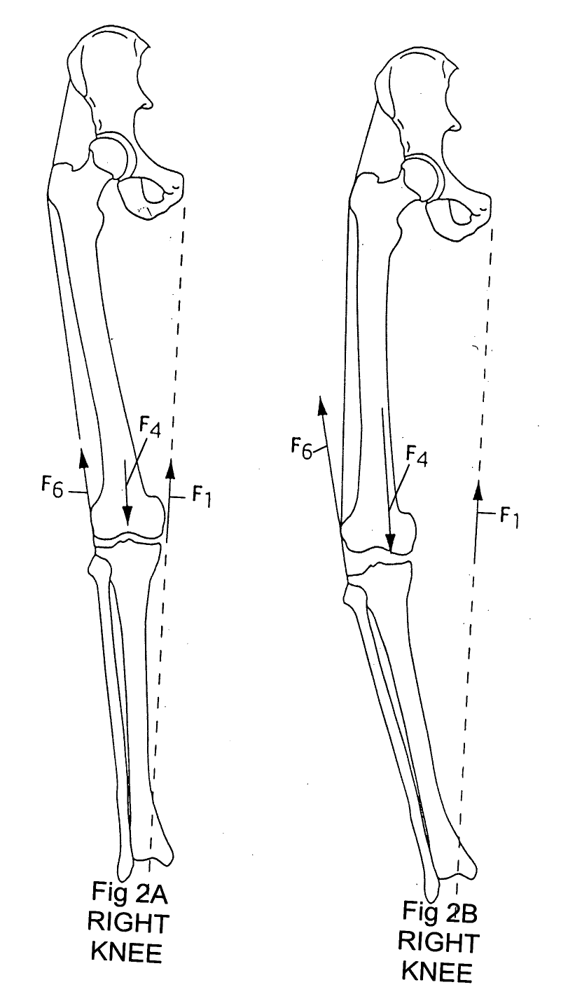

FIGS. 2A and 2B compares the forces of a normal knee alignment FIG. 2A and a knee in varus alignment FIG. 2B. The Joint Reaction Force ORF) F4 moves further medial in the medial compartment FIG. 2B and larger forces are required to balance the ADduction moment. (F6: Abductor Muscle Force; F4: Joint Reaction Force; F1: Mechanical Axis)

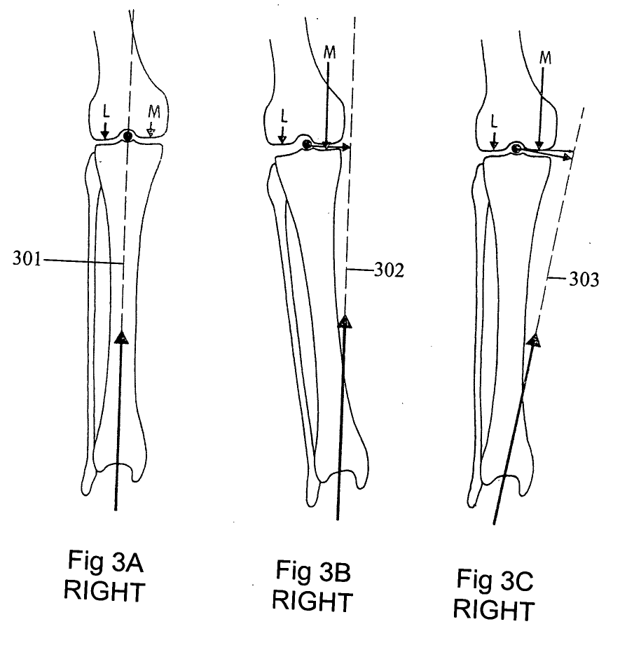

FIGS. 3A, 3B, 3C show the same forces as in FIG. 2A, 301 and FIG. 28, 302 which are static diagrams. FIG. 3C is a normal knee as in FIG. 3A dynamically loaded. Showing a dynamic ADduction moment 303 during stance

The dynamically loaded knee FIG. 3C 303 has an additional load vector occurring during normal gait that changes the moments from 3A 301 to a picture more like 3B 302.

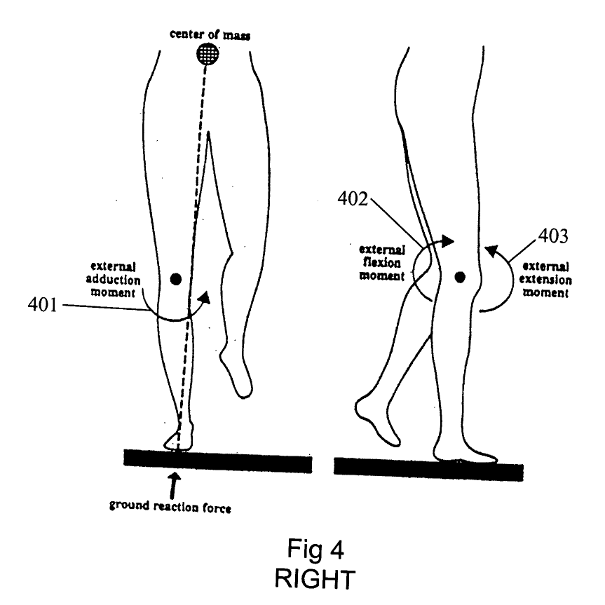

FIG. 4 shows the ADduction moment in the coronal plan 401 and the external flexion moment 402 and extension moment 403 about the knee in the sagittal plane. It has been found experimentally that these moments are not independent and that the external flexion and external extension moments affect the ADduction moment.

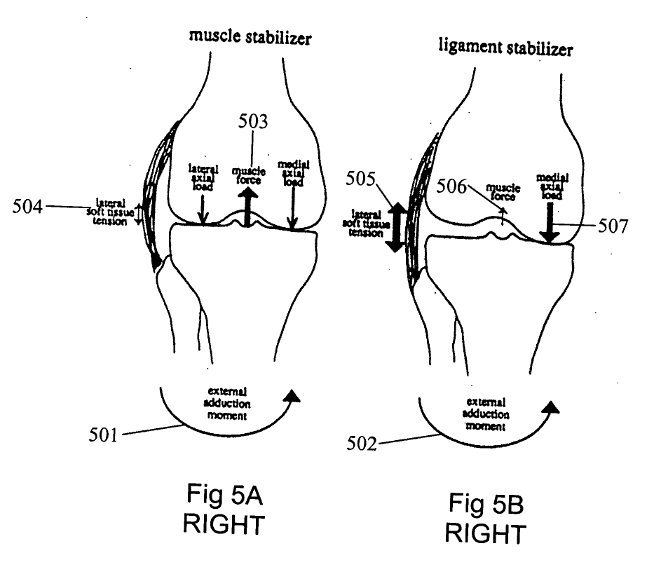

FIGS. 5A and 5B show two mechanisms that the knee can use to balance ADduction moments 501 and 502. Normally FIG. 5A the loads are shared on the medial and lateral compartments and muscle forces 503 and soft tissue tension 504 balance the moments. FIG. 5B shows a varus knee that increases in soft tissue tension 505, decreases muscle force 506 and increases the medial load 507 and shifting it more medial to balance the ADduction moment 502.

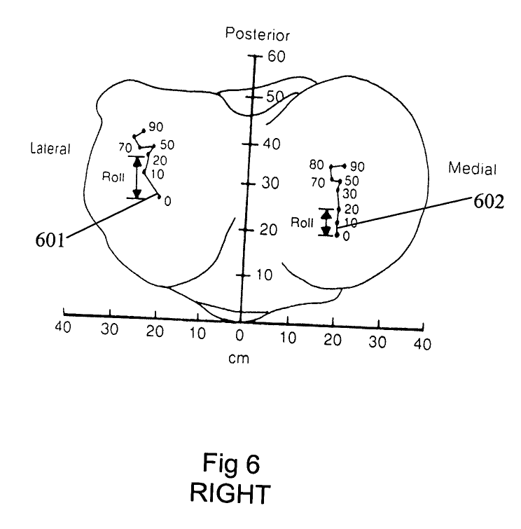

FIG. 6 shows the motion of the contact point of the femur on the tibia. This is the believed normal pattern. (Lateral compartment contact path 601; Medial Compartment contact path 602).

Variations from this pattern of pathways of the contact points 601 and 602 disrupt the biomechanics and are felt to increase joint damage. Abnormal patterns can be corrected or improved with MVCS.

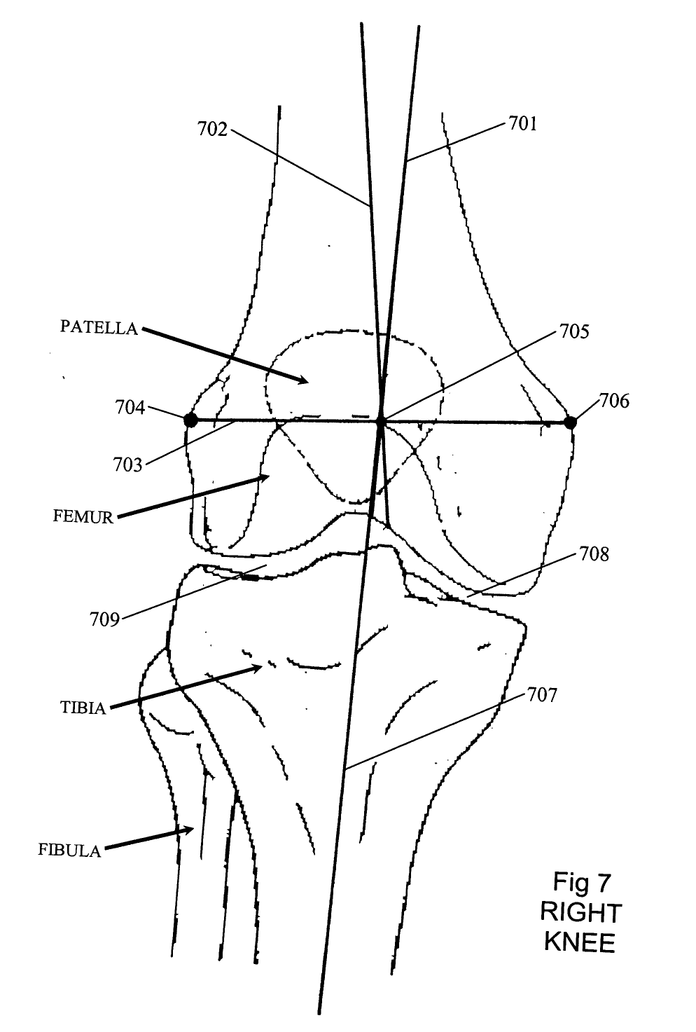

FIG. 7 shows a normal knee with weight bearing axis (WBA) 701, mechanical axis of the femur 702, trans-epicondylar axis 703, application point of muscle forces 704, axis of rotation in the coronal plane 705, application point of the reaction to BW 705, mechanical axis of the tibia 707 [Same as WBA of the tibia], Medial joint line 708, lateral joint line 709. The knee is in equilibrium. (General Anatomy for orientation is labeled: Patella, Femur, Tibia, Fibula. This is the same anatomy for FIGS. 7-17).

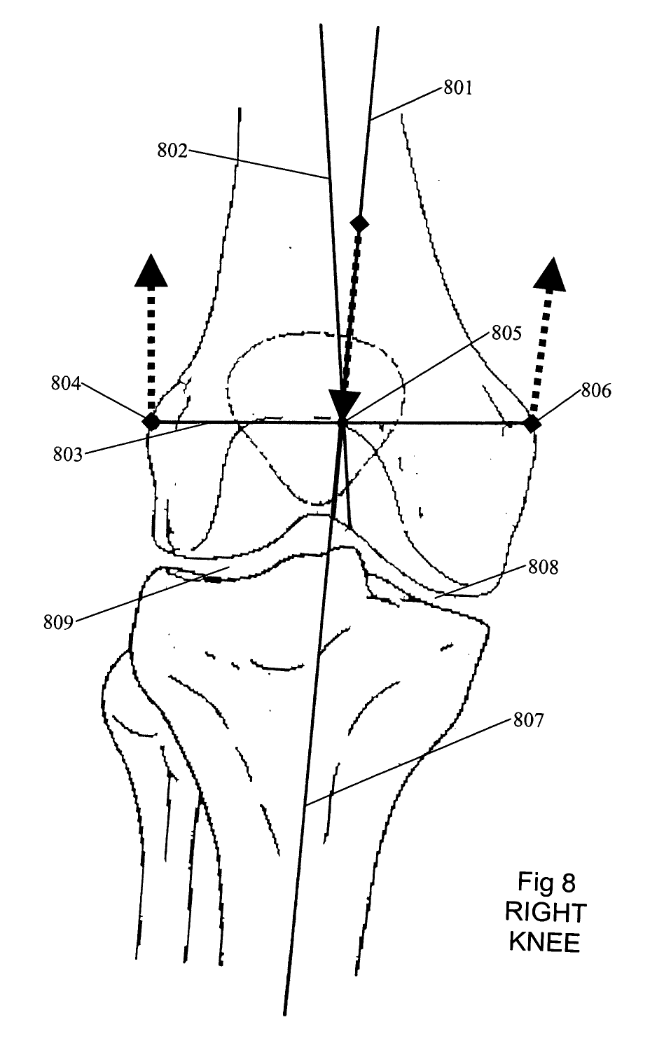

FIG. 8 shows the generally accepted forces applied to a knee joint when loaded 804 muscle pull, 805 JRF and 806 BW.

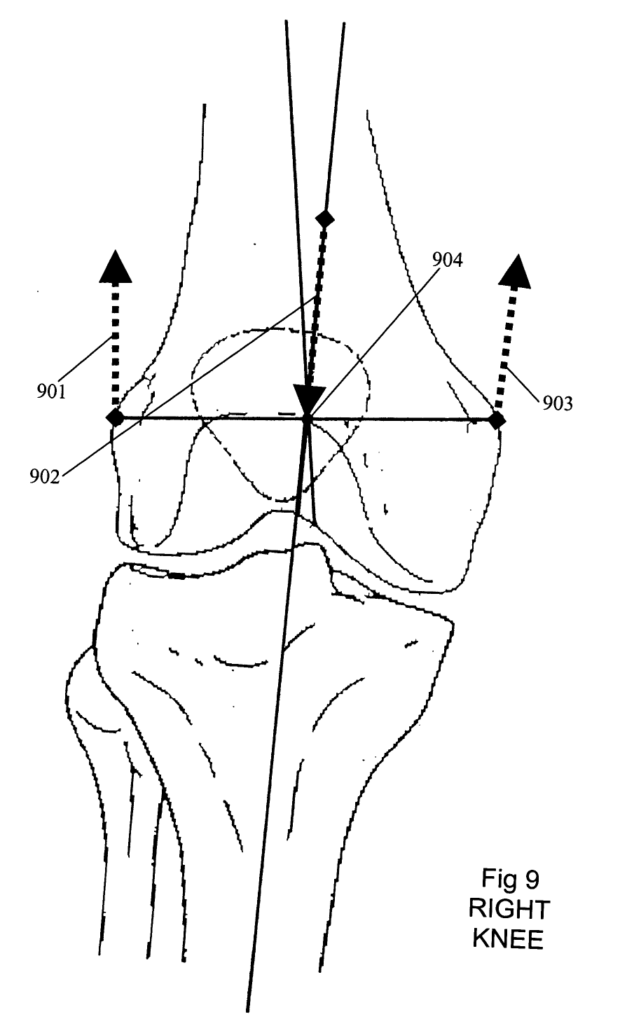

FIG. 9 shows the balanced forces. 901 Muscle forces balance 903 BW. 902 JRF is applied at the axis of rotation 904. It is balanced by an equal and opposite force from the tibia through the ground reaction force (GRF) at 904.

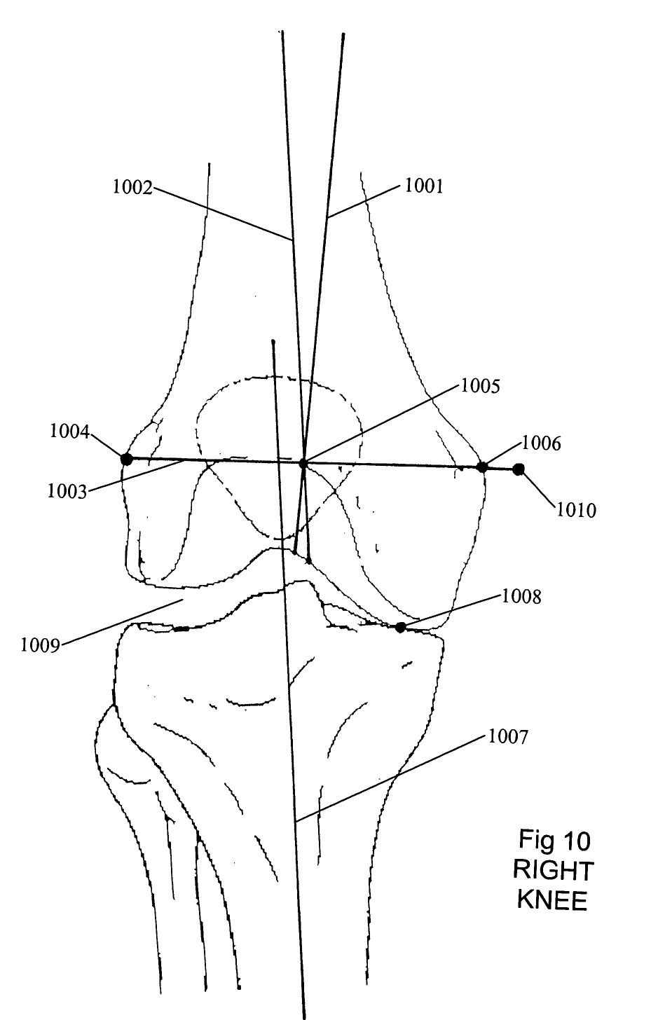

FIG. 10 shows a Varus Knee where the joint is malaligned and the joint is touching at 1008. There is joint contact at 1008 and a larger moment arm 1010. The mechanical axis of the tibia 1007 is now lateral to the coronal axis of rotation. This is thought to shift the axis 1005 lateral which changes the lengths of the moment arms.

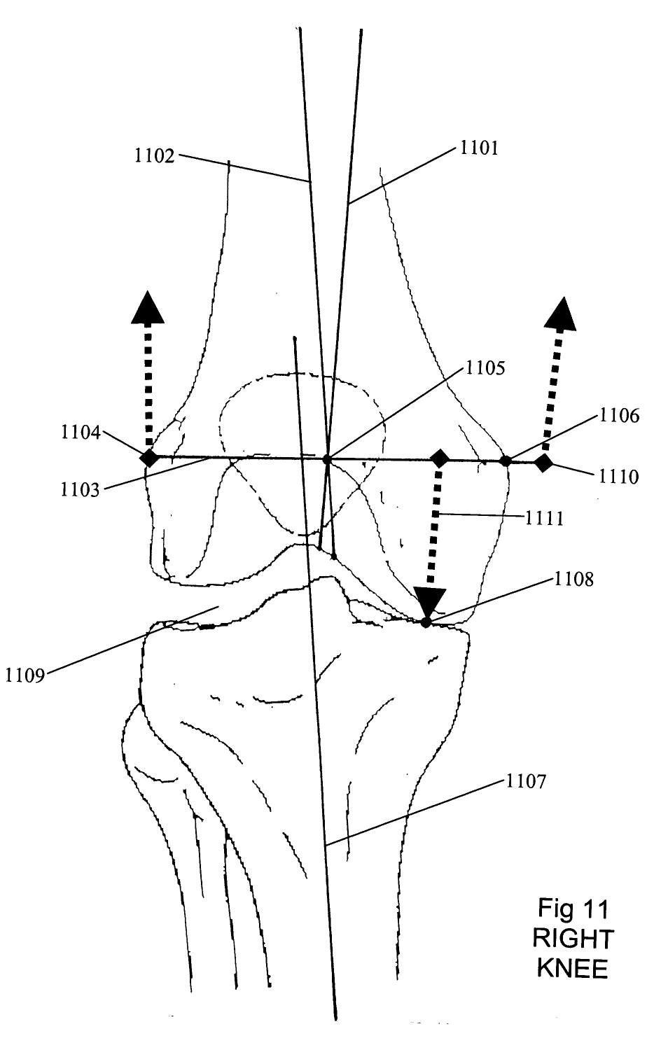

FIG. 11 shows the new forces 1104, 1110 and 1111 and the new moments (Force times moment arm length). 1104 ABductor muscle will have to increase, JRF 1108 is now shifted medial and BW moment has shifted medially and is larger.

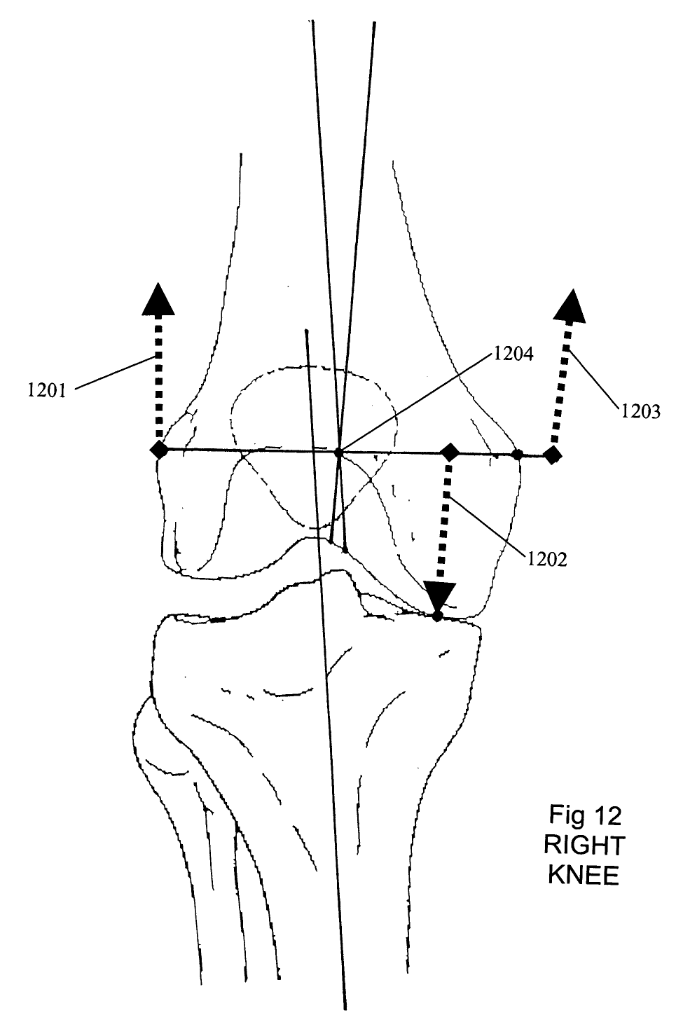

FIG. 12 shows the forces and moments independent of the other vectors 1201 Muscle forces must now be larger. 1202 is now moved medial and is larger. 1203 has a larger moment arm so it produces a larger moment. 1204 is the axis of rotation in the coronal plane.

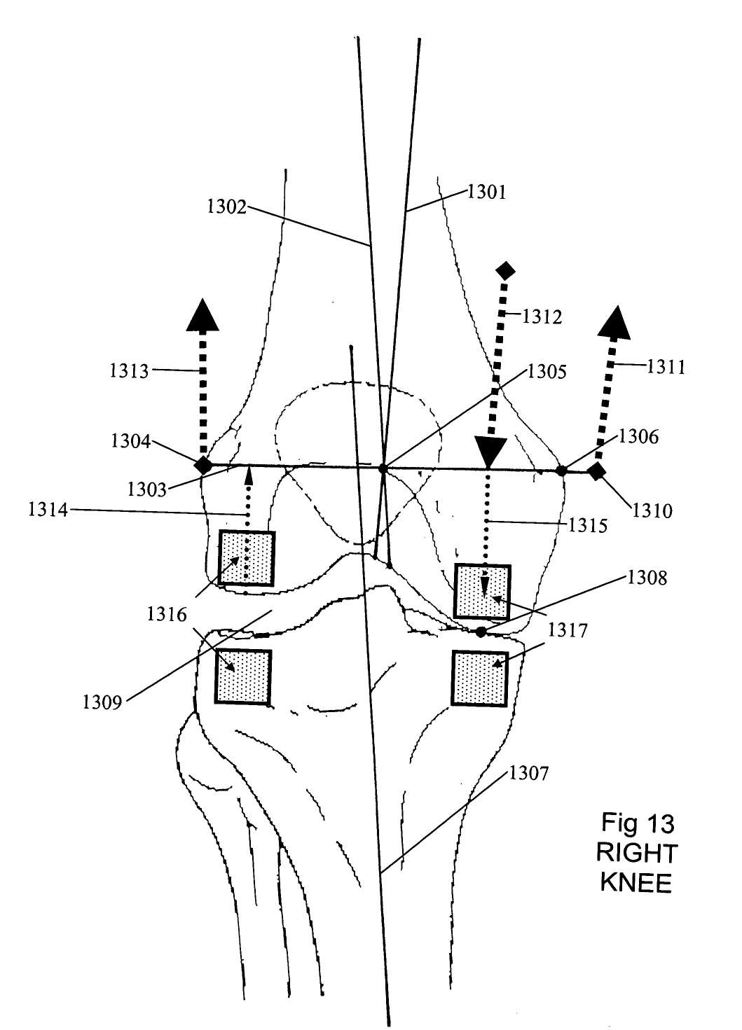

FIG. 13 shows two MVCS 1316 and 1317 implanted near the medial 1308 and lateral joint 1309 lines of the knee. They can be implanted by the TransOsseous approach (Hyde U.S. Pat. No. 6,589,521). MVCS Medial 1317 in this example is a Magnetic Array System (Hyde U.S. Pat. No. 6,387,096) that provides a substantially repulsive force. This produces an ABduction moment 1310. MVCS Lateral 1316 in this example is a simple magnet pair in attraction. This also produces an ABduction moment. The MVCS Lateral 1316 and the MVCS Medial 1317 act as a force couple in this example and reduces the Adduction 1310 moment of the varus knee. The force couple can be large enough to offload the medial compartment 1309.

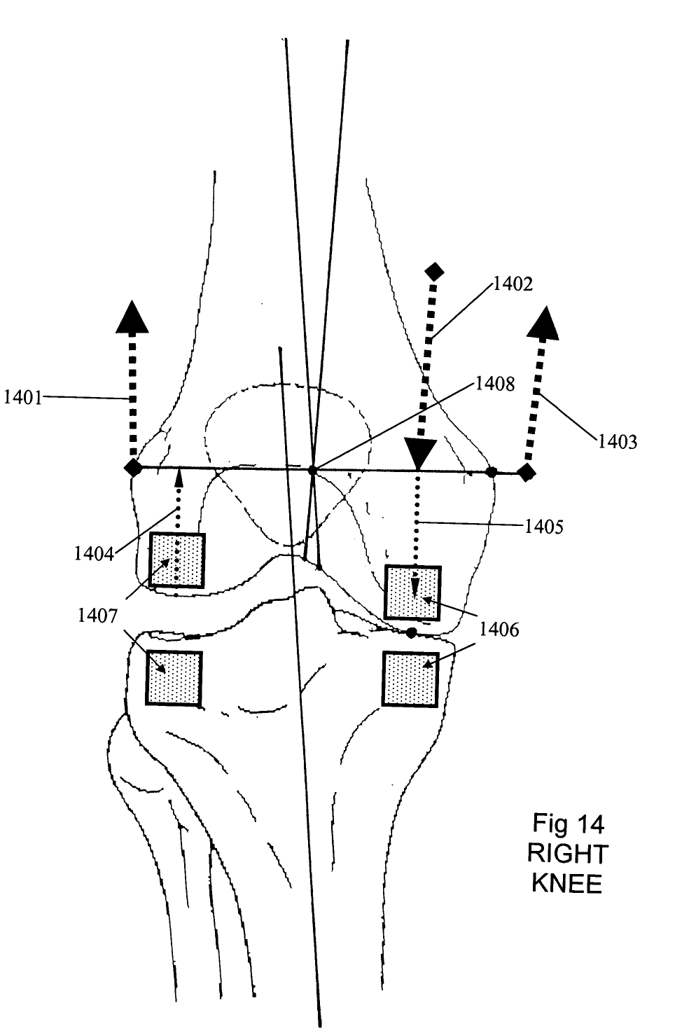

FIG. 14 shows the vectors for the varus knee 1401, 1402 and 1403 and the magnetic force couple (1404 and 1405) including the muscle tension 1401 can balance or offload (1402 and 1403.)

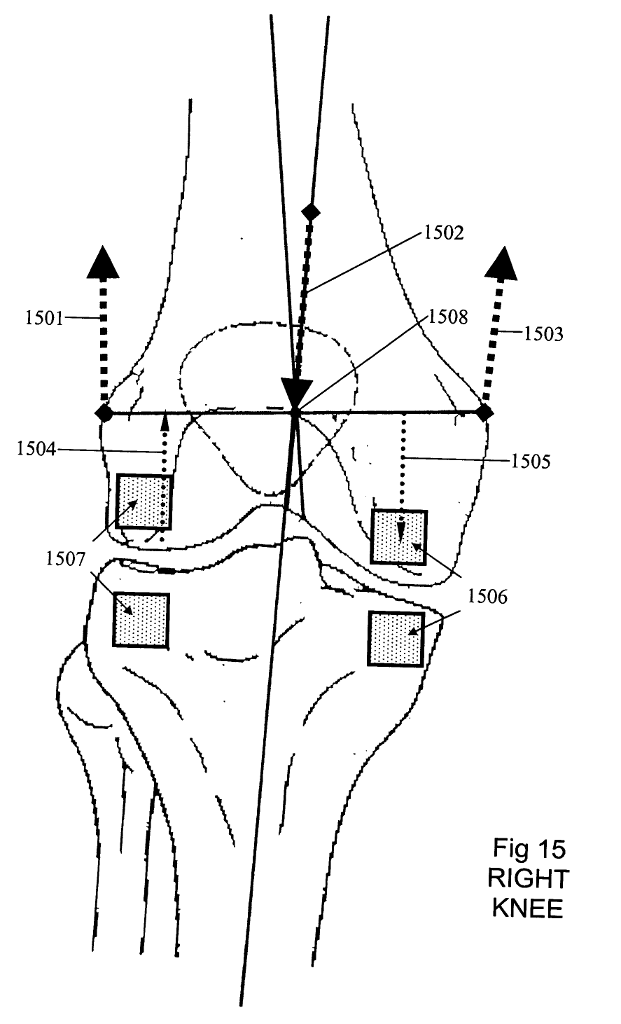

FIG. 15 shows the resultant correction of the knee with symmetrically spaced medial and lateral joint spaces due to the MVCS from a varus position to a substantially normal alignment and configuration of forces and moments.

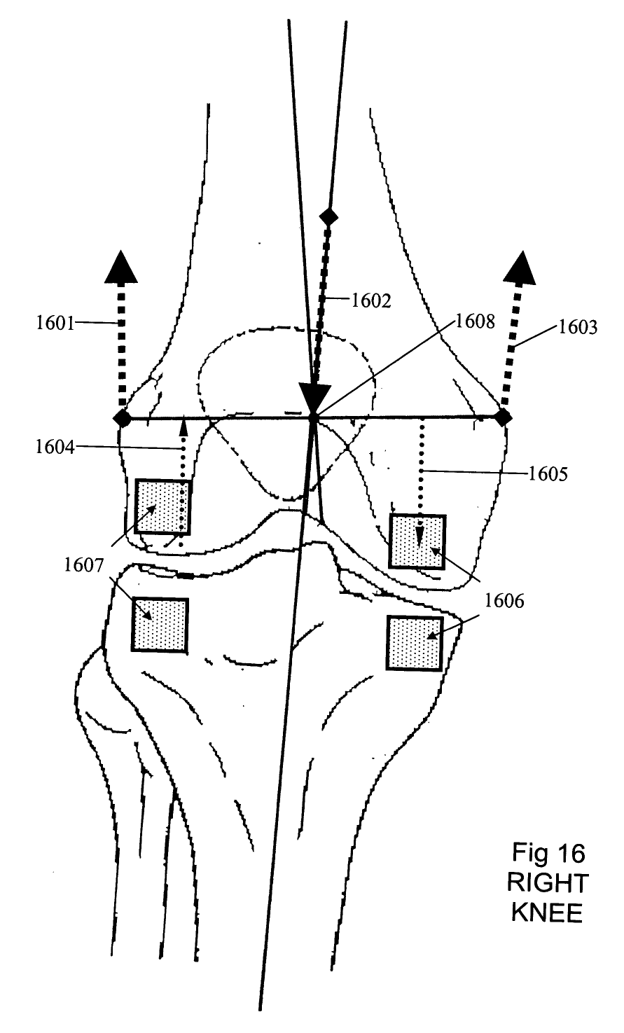

FIG. 16 shows the corrected knee with the balanced equilibrium of moments 1601, 1602 1603, 1604 and 1605 and the JRF 1602 in a neutral position.

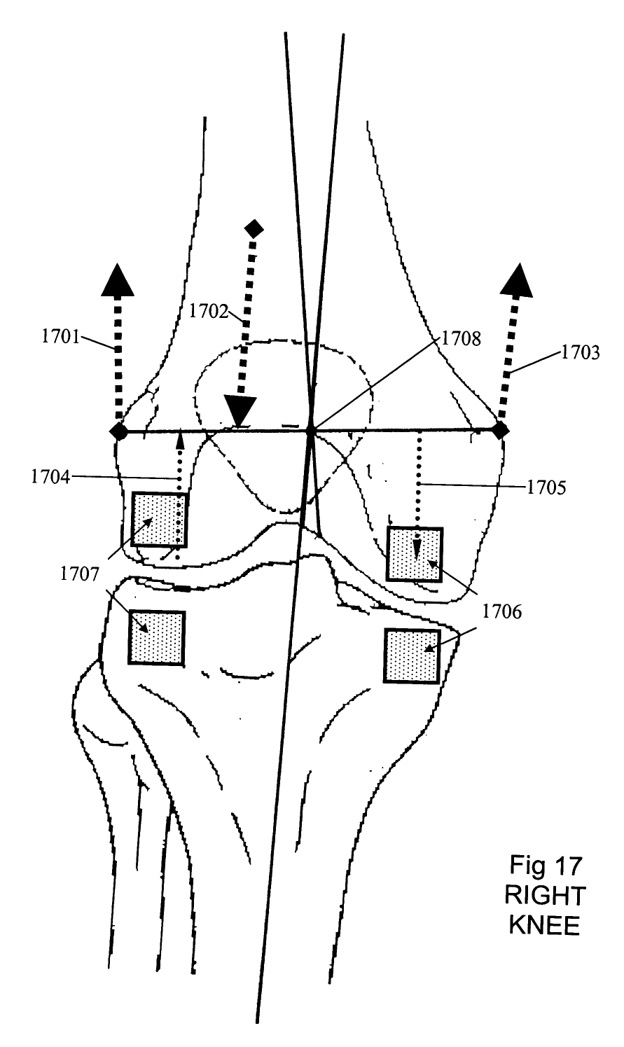

FIG. 17 shows the over-corrected knee with the JRF 1702 shifted to the lateral side of the knee by MVCS 1707 and 1706. This would effectively off load the medial joint surface and could be used as a treatment for arthritis of the medial joint space. Likewise the axis could be shifted from the lateral to the medial side to off load the lateral joint space for arthritis of the lateral joint.

MVCS can be used in any applicable positions in a joint to accurately position magnetic vectors to balance maladaptive biomechanical vectors in any plane.

Gait Lab studies using force plates and other methods can be used to calculate the ADduction moment for a patient. Any other moment can be calculated for different planes of motion. This information can be used to individualize the MVCS used and their location for individual patients. Other methods that will become available in the future for assessing gait and moments arms can also be used to determine the correct size, strength and location of the MVCS to be implanted.

The drawings and explanations in this patent application have concentrated on applications for the knee in the coronal plane and when the knee is in full extension. The MVCS can be deployed or designed such that they produce different magnetic vectors at different points of the knee range of motion from 0-150 degrees. For example the magnitude and direction of the magnetic vector can be made to be one vector when the knee is at 0-10 degrees of flexion can be very different at 80-90 degrees. It is practical to have the potential to make the vectors vary every 10 degree increment or even less if desired.

The implantation of the MVCS can be by the Transosseous Approach or any other practical method.

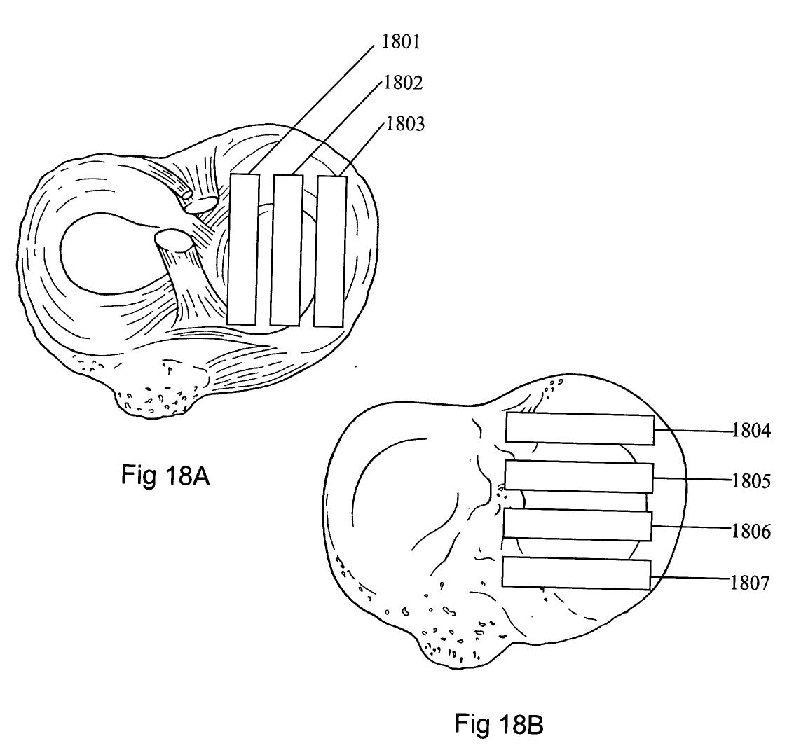

FIG. 18A shows a MVCS with rod shaped components viewed implanted in the tibia viewed from above 1801 1802 and 1803 from an anterior approach. FIG. 18B shows a MVCS with rod shaped components implanted in the tibia viewed from above 1804, 1805, 1806 and 1807 implanted from a medial approach.

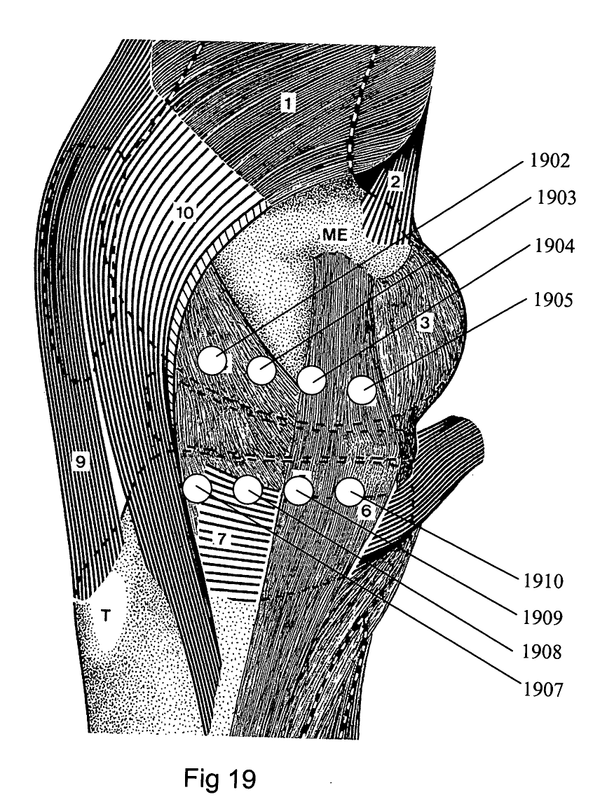

FIG. 19 shows rod shaped MVCS from a medial view similar to (1804, 1805, 1806 and 1807) in the tibia represented by 1910, 1909, 1908 and 1907 implanted from a medial approach. FIG. 19 also shows corresponding MVCS 1902, 1903, 1904 and 1905 implanted in the femur from a medial approach. The MVCS in the femur and the tibia interact to produce the desired vectors and moments.

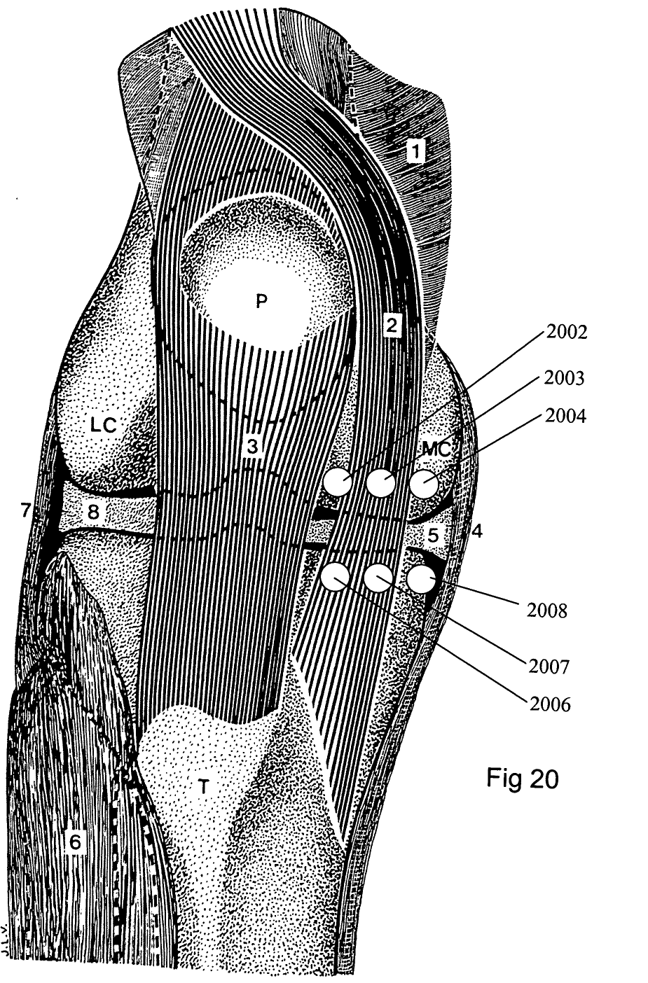

FIG. 20 shows a modular MVCS embodiment implanted from a anterior approach. 2002 2003 and 2004 are implanted in the femur from an anterior approach. 2008, 2007 and 2006 are implanted in the tibia from an anterior approach.

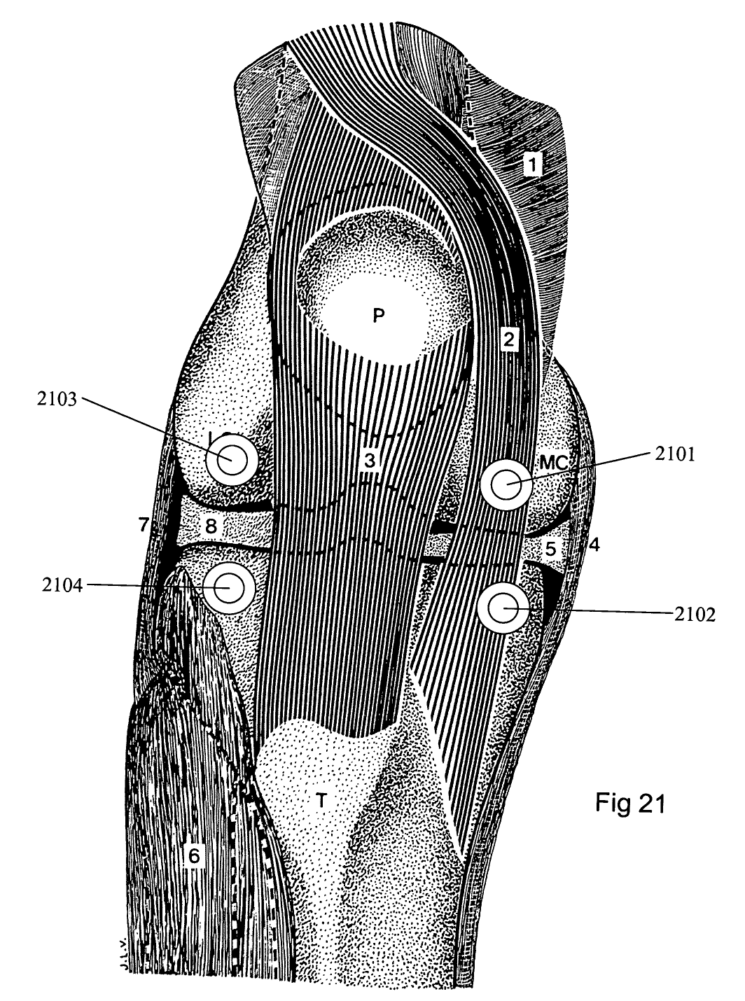

FIG. 21 shows modular MVCS 2101, 2102, 2103 and 2104 implanted from an anterior approach. This embodiment shows MVCS on both sides of the joint and correspondingly on opposed sides of a chosen mechanical axis.

Any practical placement method can be used. The MVCS can be modular so the cortical window can be small and then assembled in an enlarged space that is made through a small cortical window. The space can be made by compacting bone or removing bone or both. Implants are designed to be easily inserted and substantially easy to remove. They can be modular to aid insertion and facilitate customization of the MVCS in the OR.

1. A prosthetic assembly of magnetic field sources that are disposed at or near a joint typically on or in adjacent bones of the joint, on one side of a first mechanical axis that creates a torque or moment about a second different axis of the joint, which intersects the first mechanical axis, to decrease the joint reactive force at a joint surface or equivalently, substantially shift the first mechanical axis to a new or preferred position

2. The prosthetic assembly of claim 1 where the elements on opposite sides of the joint act in repulsion

3. The prosthetic assembly of claim 1 where the elements on opposite sides of the joint act in attraction

4. The prosthetic assembly of claim 1 where the elements on opposite sides of the joint act in repulsion and attraction

5. The prosthetic assembly of claim 1 where the magnetic elements are magnetic arrays (Hyde U.S. Pat. No. 6,387,096) that have magnetic field interactions that substantially provide stability to magnet assemblies by interaction of shaped magnetic fields

6. The prosthetic assembly of claim 1 that is placed in a joint that has acquired a maladaptive moment to help reduce the maladaptive moment towards a more normal physiological state

7. The prosthetic assembly of claim 1 where magnetic elements can be placed to function in any plane or combinations of planes, including the coronal plane, sagittal plane and axial planes of a joint

8. The prosthetic assembly of claim 1 where the magnetic elements can be used to influence any vector, vector system, moment, center or rotation and/or Instant Axis of Rotation of any joint or body segment

9. The prosthetic assembly of claim 1 where the magnetic elements can be incorporated into an implant that is implanted in a joint.

10. A prosthetic assembly of magnetic field sources that are disposed at or near a joint typically on or in adjacent bones of the joint, on at least two substantially opposed sides of a first mechanical axis that creates a torque or moment about a second different axis of the joint, which intersects the first mechanical axis, to decrease the joint reactive force at the joint surface or equivalently substantially shift the first mechanical axis to a new or preferred position

11. The prosthetic assembly of claim 10 where the elements on opposite sides of the joint act in repulsion

12. The prosthetic assembly of claim 10 where the elements on opposite sides of the joint act in attraction

13. The prosthetic assembly of claim 10 where the elements on opposite sides of the joint act in repulsion and attraction

14. The prosthetic assembly of claim 10 where the magnetic elements are magnetic arrays (Hyde U.S. Pat. No. 6,387,096) that have magnetic field interactions that substantially provide stability to magnet assemblies by interaction of shaped magnetic fields

15. The prosthetic assembly of claim 10 that is placed in a joint that has acquired a maladaptive moment to help reduce the maladaptive moment towards a more normal physiological state

16. The prosthetic assembly of claim 10 where magnetic elements can be placed to function in any plane or combinations of planes, including the coronal plane, sagittal plane and axial planes of a joint

17. The prosthetic assembly of claim 10 where the magnetic elements can be used to influence any vector, vector system, moment, center or rotation and/or Instant Axis of Rotation of any joint or body segment

18. The prosthetic assembly of claim 10 where the magnetic elements can be incorporated into an implant that is implanted in a joint.
